(19) United States
(12) Patent Application Publication
(10) Pub. No.: US 2008/0051403 A1
(43) Pub. Date: Feb. 28, 2008

(54) Treatment For Alzheimer's Disease And Related Conditions

(76) Inventor: Jose Luis Castro Pineiro, Bishops Stortford (GB)

Correspondence Address: MERCK AND CO., INC, P O BOX 2000, RAHWAY, NJ, 07065-0907, US

(21) Appl. No.: 10/581,291

(22) PCT Filed: Dec. 14, 2004
(86) PCT No.: PCT/GB04/05234

§ 371 Date: Jun. 1, 2006

(30) Foreign Application Priority Data
Dec. 18, 2003 (GB) 0329275.2

(51) Int. Cl. A61K 31/4155; A61K 31/404; A61P 25/00; A61P 25/28; A61P 43/00

(52) U.S. Cl. 514/236.5; 514/278

(57) ABSTRACT

This invention provides the combination of a growth hormone secretagogue and a p38 kinase inhibitor for use in treatment or prevention of a disease associated with deposition of Aβ in the brain.

This invention relates to the use of methods and materials for therapeutic treatment of the human body. In particular, it provides methods of treating diseases associated with the deposition of β-amyloid in the brain, such as Alzheimer's disease, or of preventing or delaying the onset of dementia associated with such diseases.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD. Various interventions in the plaque-forming process have been proposed as therapeutic treatments for AD (see, for example, Hardy and Selkoe, Science, 297 (2002), 353-6).

Other dementing conditions associated with deposition of Aβ in the brain include cerebral amyloid angiopathy, hereditary cerebral haemorrhage with amyloidosis, Dutch-type (HCHWA-D), multi-infarct dementia, dementia pugilistica and Down syndrome.

Carro et al, in Nature Medicine, 8 (2002), 1390-7, disclose that subcutaneous administration of insulin-like growth factor 1 (IGF-1) causes a reduction in the cerebral A burden in certain rodents. However, some evidence suggests that interference with brain Aβ levels may be linked with adverse effects in humans (see New Scientist, Feb. 1, 2003, 35-7).

Growth hormone has been proposed for use in treatment of AD. Thus, U.S. Pat. No. 4,902,680 advocates the administration of growth hormone to patients in the advanced stages of AD, while WO 00/13650 discloses that increased levels of growth hormone in the brain provide a neuroprotective effect, and in particular can rescue neurons that would otherwise die as a result of an insult such as that associated with a neurodegenerative disease such as AD. The injection of growth hormone into the brain is contemplated.

Growth hormone secretagogues (GHSs) are compounds which, when administered to an animal (such as a human), stimulate or increase the release of endogenous growth hormone in the animal. Their mode of action and clinical utilities are reviewed by Ankersen et al, Drug Discovery Today, 4 (1999), 497-506; Casanueva and Dieguez, TEM, 10 (1999), 30-8; Smith et al, ibid., 10 (1999), 128-35; Betancourt and Smith, J. Anti-Aging Med., 5 (2002), 63-72; and Ghigo et al, ibid., 5 (2002), 345-56, but there is no mention of treating AD or any other neurodegenerative condition. Patents and patent applications disclosing compounds which are GHSs include U.S. Pat. No. 5,767,124, U.S. Pat. No. 5,536,716, WO 94/13696, EP 0615977B, US 5,578,593; WO 01/04119, WO 98/25897, WO 98/10653, WO 97/36873, WO 97/34604, WO 97/15574, WO 97/11697, WO 96/32943, WO 96/13265, WO 96/02530, WO 95/34311, WO 95/14666, WO 95/13069, WO 94/19367, WO 94/05634 and WO 92/16524 (all assigned to Merck & Co., Inc.); EP 1002802A, EP 0995748A, WO 98/58948, WO 98/58947 and WO 97/24369 (all assigned to Pfizer Inc.); WO 01/34593, WO 00/26252, WO 00/01726, WO 99/64456, WO 99/58501, WO 99/36431, WO 98/58950, WO 98/08492, WO 98/03473, WO 97/40071, WO 97/40023, WO 97/23508, WO 97/00894, WO 96/24587, WO 96/24580, WO 96/22997, WO 95/17423 and WO 95/17422 (all assigned to Novo Nordisk A/S); WO 96/15148 (Genentech Inc.); WO 97/22620 (Deghenghi); WO 02/32888, WO 02/32878, WO 00/49037, WO 00/10565 and WO 99/08699 (all assigned to Eli Lilly and Co.); WO 02/057241 and WO 02/056873 (both assigned to Bayer Corp.); and WO 01/85695, WO 00/54729 and WO 00/24398 (all assigned to Bristol-Myers Squibb Co.). The compounds are recommended for use in promoting the growth of food animals, and in humans for treating physiological or medical conditions characterised by a deficiency in growth hormone secretion, and medical conditions which are improved by the anabolic effects of growth hormone. In some of the above-listed disclosures, the list of treatable conditions includes AD.

The compound disclosed in the aforementioned U.S. Pat. No. 5,767,124 has been the subject of a number of clinical trials in therapeutic fields unrelated to AD (see, for example, Murphy et al, J. Bone Miner. Res., 14, (1999), 1182-8; Chapman et al, J. Clinical Endocrinology and Metabolism, 81, (1996), 4249-57; ibid., 82, (1997), 3455-63; and Svensson et al, ibid., 83, (1998), 362-9).

Mitogen activated protein kinases (MAPKs) are a family of serine/threonine protein kinases that play a critical role in transducing multiple signals from the cell surface to the nucleus in all eukaryotic species. A subgroup of MAPKs includes the stress-activated protein kinases, of which p38 is an example. p38 exists α, β, γ and δ isoforms and has a major role in the production and action of a number of pro-inflammatory mediators. Consequently, inhibitors of p38 kinase have been suggested for use in treating a variety of inflammatory disorders such as arthritis in its various forms and autoimmune diseases. p38, and p38α in particular, has also been implicated in the signalling pathways leading to apoptosis in neurodegenerative diseases such as Parkinson's disease, Alzheimer's disease, Huntingdon's disease, amyotrophic lateral sclerosis and ischaemia (see, for example, Harper and Wilkie, Expert Opin. Ther. Targets, 2003, 7, 187-200; Dalrymple, J. Mol. Neurosci., 2002, 19, 295-9; and Zhu et al, Neurosignals, 2002, 11, 270-81). Thus, p38 kinase inhibitors have been suggested as a means of controlling the neurotoxic effects of intracerebral Aβ. However, there has hitherto been no suggestion of any role for p38 kinase inhibitors in controlling the accumulation of Aβ in the brain.

According to the invention, there is provided the combination of a growth hormone secretagogue and a p38 kinase inhibitor for use in treatment or prevention of a disease associated with deposition of Aβ in the brain.

Also according to the invention, there is provided a method of treatment or prevention of a disease associated with deposition of Aβ in the brain comprising administering to a subject in need thereof a therapeutically effective amount of a growth hormone secretagogue (GHS) in combination with a therapeutically effective amount of a p38 kinase inhibitor.

Said disease is typically Alzheimer's disease, cerebral amyloid angiopathy, multi-infarct dementia, dementia pugilistica or Down syndrome, preferably Alzheimer's disease.

The invention further provides a method of treating, preventing or delaying the onset of dementia associated with Alzheimer's disease, cerebral amyloid angiopathy, multi-infarct dementia, dementia puglistica or Down syndrome comprising administering to a patient in need thereof a therapeutically effective amount of a growth hormone secretagogue in combination with a therapeutically effective amount of a p38 kinase inhibitor.

As used herein, the expression “in combination with” requires that therapeutically effective amounts of both a GHS and an agent which modifies the production or processing of Aβ in the brain (hereinafter termed an “amyloid modifier”) are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day The separate species may be administered by the same route or by different routes, e.g. one species orally and the other parenterally, although oral administration of both species is preferred, where possible. When the amyloid modifier is an antibody, it will typically be administered parenterally and separately from the GHS.

According to a further aspect of the invention there is provided a pharmaceutical composition comprising, in a pharmaceutically acceptable carrier, a growth hormone secretagogue and a p38 kinase inhibitor.

The invention further provides the use, for the manufacture of a medicament for treatment or prevention of a disease associated with deposition of Aβ in the brain, of a growth hormone secretagogue and a p38 kinase inhibitor.

Said disease is typically Alzheimer's disease, cerebral amyloid angiopathy, multi-infarct dementia, dementia pugilistica or Down syndrome, preferably Alzheimer's disease.

The GHS and p38 kinase inhibitor act synergistically in promoting the clearance of Aβ from the brain. Therefore, in a further aspect the invention provides a method for retarding, arresting or preventing the accumulation of Aβ in the brain comprising administering to a subject in need thereof a therapeutically effective amount of a growth hormone secretagogue in combination with a therapeutically effective amount of a p38 kinase inhibitor. Clearance of Aβ from the brain following administration of the relevant compounds may be evidenced by an increase in the level of soluble Aβ in the cerebrospinal fluid and/or the plasma. Alternatively (or additionally), imaging techniques such as magnetic resonance imaging, positron emission tomography, single photon emission computed tomography and multiphoton microscopy may be employed to monitor the extent of Aβ deposition in the brain (see, for example, Bacskai et al., J. Cereb. Blood Flow Metab., 22 (2002), 1035-41).

In one embodiment of the invention, the GHS and p38 kinase inhibitor are administered to a patient suffering from AD, cerebral amyloid angiopathy, HCHWA-D, multi-infarct dementia, dementia pugilistica or Down syndrome, preferably AD.

In an alternative embodiment of the invention, the GHS and p38 kinase inhibitor are administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the GHS and p38 kinase inhibitor are advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, GHS and p38 kinase inhibitor are administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment with the compound of formula I or a pharmaceutically acceptable salt thereof, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

The invention further provides a kit comprising a first medicament comprising a GHS and a second medicament comprising a p38 kinase inhibitor together with instructions for administering said medicaments sequentially or simultaneously to a patient suffering from AD, age-related cognitive decline, MCI, cerebral amyloid angiopathy, HCHWA-D, multi-infarct dementia, dementia pugilistica or Down syndrome.

The GHS used in the invention may be any compound which has the property of stimulating or enhancing secretion of endogenous growth hormone when administered to a subject, for example any of the compounds disclosed in the patents and patent applications listed above. However, preference is given to compounds which are suitable for oral administration.

A first class of GHSs suitable for use in the invention is that disclosed in WO 94/13696, in particular the subset thereof disclosed in EP 0615977B, the disclosure of which is incorporated herein by reference. Preferred examples of GHSs within this class include the compound of formula I:

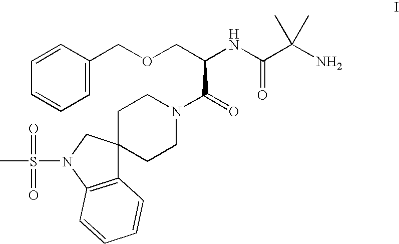

named as N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide, and pharmaceutically acceptable salts thereof, in particular the methanesulfonate salt thereof, which may be prepared as described in U.S. Pat. No. 5,767,124.

A second class of GHSs suitable for use in the invention is that disclosed in U.S. Pat. No. 5,578,593, the disclosure of which is incorporated herein by reference. Preferred example of GHSs within this class include the compound of formula II:

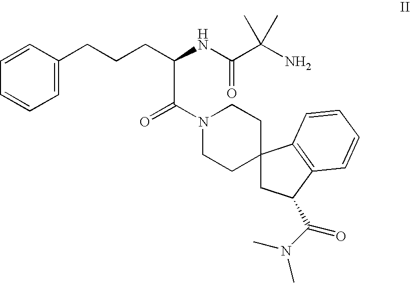

and pharmaceutically acceptable salts thereof, which may be prepared as described in U.S. Pat. No. 5,578,593.

A third class of GHSs suitable for use in the invention is that disclosed in WO 92/16524, the disclosure of which is incorporated herein by reference. Preferred example of GHSs within this class include the compounds of formulae III and IV:

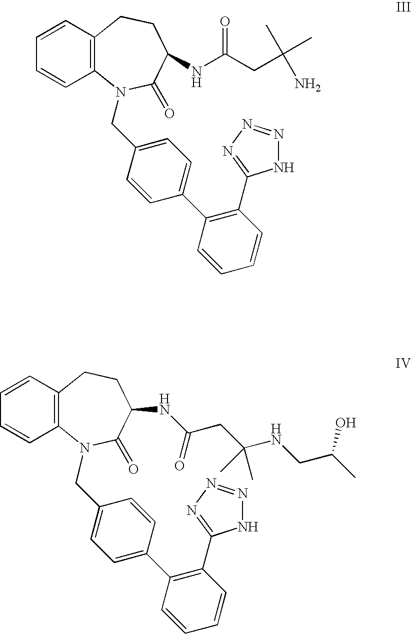

and pharmaceutically acceptable salts thereof, in particular the trifluoroacetate salts thereof, which may be prepared as described in WO 92/16524.

A fourth class of GHSs suitable for use in the invention is that disclosed in WO 97/23508, the disclosure of which is incorporated herein by reference. Preferred examples of GHSs within this class include the compound of formula V, also known as NN703:

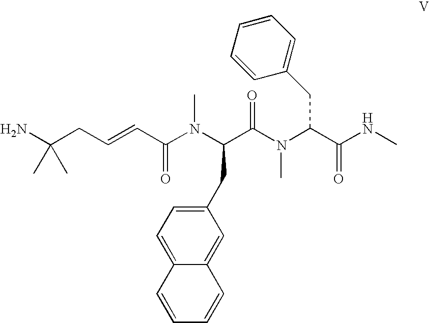

and pharmaceutically acceptable salts thereof, which may be prepared as described in WO 99/64456.

A fifth class of GHSs suitable for use in the invention is that disclosed in WO 97/24369, the disclosure of which is incorporated herein by reference. Preferred examples of GHSs within this class include the compound of formula VI:

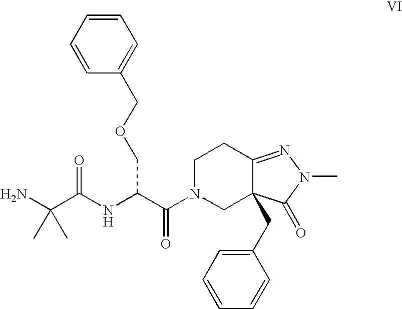

named as 2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]-isobutyramide, and pharmaceutically acceptable salts thereof, in particular the L-tartrate salt, also known as capromorelin, which may be prepared as described in WO 97/24369 and in Carpino et al, Bioorg. Med. Chem., 11 (2003), 581-90.

A sixth class of GHSs suitable for use in the invention is that disclosed in WO 98/58947, the disclosure of which is incorporated herein by reference. Preferred examples of GHSs within this class include the compound of formula VII:

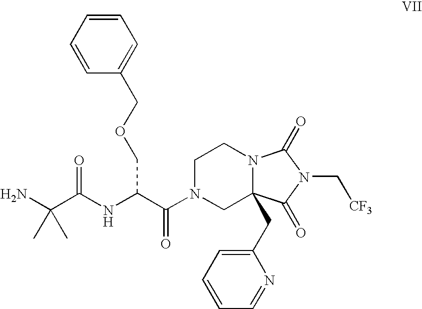

and pharmaceutically acceptable salts thereof, which may be prepared as described in WO 98/58947.

A seventh class of GHSs suitable for use in the invention is that disclosed in WO 99/08699, the disclosure of which is incorporated herein by reference. Preferred examples of GHSs within this class include the compound of formula VIII:

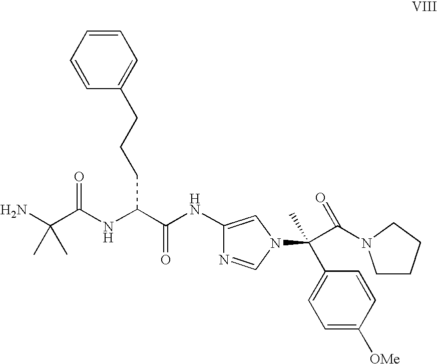

and pharmaceutically acceptable salts thereof, which may be prepared as described in WO 99/08699 and WO 02/32878.

Further GHSs suitable for use in the invention include the compound of formula IX;

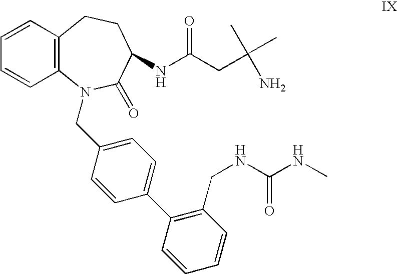

and pharmaceutically acceptable salts thereof, which may be prepared as described in De Vita et al, J. Med. Chem., 41 (1998), 1716-28, and the compound of formula X:

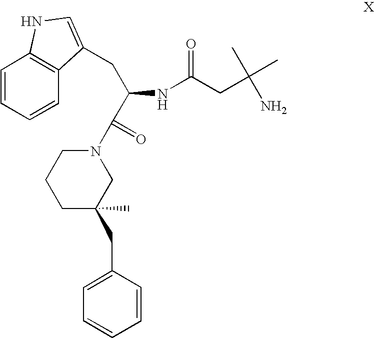

and pharmaceutically acceptable salts thereof, which may be prepared as described in Yang et al, J. Med. Chem., 41 (1998), 2439-41.

The p38 kinase inhibitor may in principle be any compound known to inhibit p38 kinase. Assays to identify such compounds are well known in the art and are described, for example, in WO 02/058695 and WO 01/64679. The p38 kinase inhibitor is preferably selective for the α isoform, and in particular selective for the α isoform over the γ and δ isoforms. Preference is also given to compounds suitable for oral administration. Examples of suitable p38 kinase inhibitors include minocycline (Lin et al, Neurosci. Lett., 2001, 315, 61-4); doramapimod (WO 02/07772); SB-239063 (Ward et al, Pharmaceutical Research, 2001, 18, 1336-44); the compounds identified as VX-702 (Vertex Pharmaceuticals Inc.), SCIO-469 and SCIO-323 (Scios Inc.), 681323 (GlaxoSmithKIine plc), AMG-548 (Amgen Inc.) and CT-8730 (Celltech Group plc); and compounds disclosed in patents such as WO 97/05877, WO 97/05878, WO 97/12876, WO 97/16442, WO 97/47618, WO 00/31065, WO 01/01988, WO 01/00208, WO 00/69848, WO 01/22965, WO 00/06563, WO 01/42241, WO 02/058695, WO 01/64679, US 03/17821, WO 03/000682, US 03/14777. Preferred p38 kinase inhibitors include the compounds of formula XI:

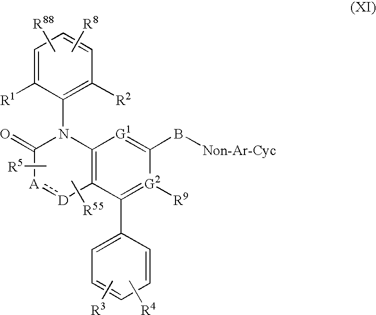

or pharmaceutically acceptable salts thereof, wherein

Non-Ar-Cyc is

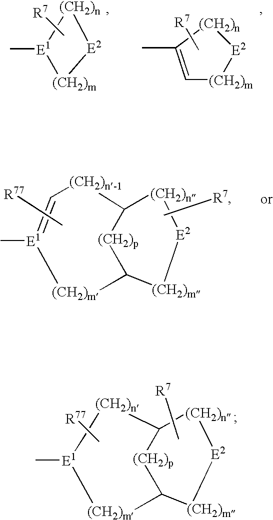

A is N, O, NH, CH₂, or CH;

B is —C₁₋₆alkyl-, —C₀₋₃alkyl-O—C₀₋₃alkyl-, —C₀₋₃alkyl-NH—C₀₋₃alkyl-, —C₀₋₃alkyl-NH—C₃₋₇cycloalkyl-, —C₀₋₃alkyl-N(C₀₋₃alkyl)-C(O)—C₀₋₃alkyl-, —C₀₋₃alkyl-NH—SO₂—C₀₋₃alkyl-, —C₀₋₃alkyl-, —C₀₋₃alkyl-S—C₀₋₃alkyl-, —C₀₋₃alkyl-SO₂—C₀₋₃alkyl-, —C₀₋₃alkyl-PH—C₀₋₃alkyl-, —C₀₋₃alkyl-C(O)—C₀₋₃alkyl, or a direct bond;

D is CH, CH₂, N, or NH; optionally A and D are bridged by —C₁₋₄alkyl- to form a fused bicyclo ring with A and D at the bicyclo cusps;

E¹ is CH, N, or CR⁶; or B and E¹ form —CH═C<;

E² is CH₂, CHR, C(OH)R NH, NR, O, S, —S(O)—, or —S(O)₂—;

G¹ is N, CH, or C(C₁₋₃alkyl);

G² is N, CH, or C(C₁₋₃alkyl);

R, R⁷ and R⁷⁷ each independently is hydrogen, C₁₋₆alkyl-group, C₂₋₆alkenyl-group, C₄₋₆cycloalkyl-C₀₋₆alkyl-group, N(C₀₋₄alkyl)(C₀₋₄alkyl)-C₁₋₄alkyl-N(C₀₋₄alkyl)-group, —N(C₀₋₄alkyl)(C₀₋₄alkyl) group, C₁₋₃alkyl-CO—C₀₋₄alkyl-group, C₀₋₆alkyl-O—C(O)—C₀₋₄alkyl-group, C₀₋₆alkyl-C(O)—O—C₀₋₄alkyl-group, N(C₀₋₄alkyl)(C₀₋₄alkyl)-(C₀₋₄alkyl)C(O)(C₀₋₄alkyl)-group, phenyl-C₀₋₄alkyl-group, pyridyl-C₀₋₄alkyl-group, pyrimidinyl-C₀₋₄alkyl-group, pyrazinyl-C₀₋₄alkyl-group, thiophenyl-C₀₋₄alkyl-group, pyrazolyl-C₀₋₄alkyl-group, imidazolyl-C₀₋₄alkyl-group, triazolyl-C₀₋₄alkyl-group, azetidinyl-C₀₋₄alkyl-group, pyrrolidinyl-C₀₋₄alkyl-group, isoquinolinyl-C₀₋₄alkyl-group, indanyl-C₀₋₄alkyl-group, benzothiazolyl-C₀₋₄alkyl-group, any of the groups optionally substituted with 1-6 substituents, each substituent independently being —OH, —N(C₀₋₄alkyl)(C₀₋₄alkyl), C₁₋₄alkyl, C₁₋₆alkoxyl, C₁₋₆alkyl-CO—C₀₋₄alkyl-, pyrrolidinyl-C₀₋₄alkyl-, or halogen;

or R⁷ together with a bond from an absent ring hydrogen is ═O;

n′+n″=n;

m′+m″=m;

n is 1, 2, 3, or 4;

m is 0, 1, 2, 3, or 4;

n+m is 2, 3, 4, 5, or 6;

p is 0, 1, 2, or 3;

R¹, R², R³, R⁴, and R⁶ are each independently halogen, C₀₋₄alkyl, —C(O)—O(C₀₋₄alkyl), or —C(O)—N(C₀₋₄alkyl)(C₀₋₄alkyl);

R⁵ and R⁵⁵ independently is H, CH₃, CH₂CH₃, or absent;

R⁸⁸ and R⁸ each is independently —CN, —C₀₋₄alkyl, —C(O)—N(C₀₋₄alkyl)(C₀₋₄alkyl), —C(O)—O—C₀₋₄alkyl or 1,3-dioxolan-2-yl-C₀₋₄alkyl-;

R⁹ is —C₀₋₄alkyl, or absent; and

any alkyl is optionally substituted with 1-6 independent halogen or —OH.

In a particular embodiment, Non-Ar-Cyc is

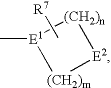

### A is CH, D is CH and G²is N.

Such compounds may be prepared as described in WO 02/058695.

In a particularly preferred embodiment of the invention, the GHS is the methanesulfonate salt of N-[1(R)-[(1,2-dihydro-1-methanesulfonyispiro[3H-indole-3,4′-piperidin]-1′-yl)carbonyl]-2-(phenylmethyloxy)ethyl]-2-amino-2-methylpropanamide and the p38 kinase inhibitor is the compound of formula XI.

Depending on whether they are to be administered together or separately, the GHS and p38 kinase inhibitor are typically supplied as single or multiple pharmaceutical compositions comprising the active species and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and poly(ethylene glycol), and other pharmaceutical diluents, e.g. water, to form a homogeneous preformulation composition containing one or both active species, or pharmaceutically acceptable salts thereof. When referring to these preformulation compositions as homogeneous, it is meant that the active species is or are dispersed evenly throughout the composition so that the composition may be readily subdivided into equally effective unit dosage forms such as tablets, pills and capsules. This preformulation composition is then subdivided into unit dosage forms of the type described above, generally containing from 0.01 to about 500 mg of the active species. Typical unit dosage forms contain from 0.05 to 100 mg, for example 0.05, 0.1, 0.5, 1, 2, 5, 10, 25, 50 or 100 mg, of the active species. Tablets or pills of the pharmaceutical composition(s) can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the pharmaceutical compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxyinethylcellulose, methylcellulose, poly(ethylene glycol), poly(vinylpyrrolidone) and gelatin.

Pharmaceutical compositions suitable for oral administration are preferred.

For treatment or prevention of AD, the GHS and p38 kinase inhibitor may be dosed at the levels which are effective for the original purposes of the separate compounds. Thus, the GHS will typically be dosed at levels known to provide increased secretion of endogenous growth hormone in a human subject, and the p38 kinase inhibitor at levels known to cause significant inhibition of the PDE4 enzyme in humans. In many cases, these dosage levels are available from the published literature, but otherwise are readily determined by standard clinical methods.

The frequency of dosing of the relevant compounds (e.g. once, twice, three times or four times per day) may be selected according to the pharmacokinetic profiles of the compounds concerned.

In the case of the preferred GHS of formula I, doses of about 0.01 to 5.0 mg/kg per day, preferably about 0.05 to 2.5 mg/kg per day, more preferably about 0.1 to 1.0 mg/kg of body weight per day, may be contemplated. In particular, a dose equivalent to 5 mg, 10 mg or 25 mg of the free base may be administered orally once daily to a patient.

In the case of the p38 kinase inhibitor of formula XI, doses of about 0.01 to 5.0 mg/kg per day may be contemplated.

In the case of doramapimod, a dose of about 30 mg per person once or twice daily may be contemplated.

In a further aspect, the invention provides a pharmaceutical composition comprising, in a pharmaceutically acceptable carrier, a compound of formula I or a pharmaceutically acceptable salt thereof and a compound of formula XI or a pharmaceutically acceptable salt thereof. Preferably the compound of formula I is in the form of the methanesulfonate salt. Preferably, the pharmaceutical composition is in a unit dose form suitable for oral administration, such as a tablet or a capsule.

1-10. (canceled)

11. A method for the treatment or prevention of a disease associated with deposition of Aβ in the brain comprising administering to a subject in need thereof a therapeutically effective amount of a growth hormone secretagogue or a pharmaceutically acceptable salt thereof and a therapeutically effective amount of a p38 kinase inhibitor or a pharmaceutically acceptable salt thereof.

12. The method of claim 11 wherein the disease is slected from Alzheimer's disease, age-related cognitive decline, mild cognitive impairment, cerebral amyloid angiopathy, multi-infarct dementia, dementia pugilistica and Down syndrome.

13. The method of claim 12 wherein the disease is Alzheimer's disease.

14. The method of claim 12 wherein the disease is mild cognitive impairment.

15. The method of claim 14 wherein the patient possesses one or more risk factors for developing Alzheimer's disease selected from: a family history of the disease; a genetic predisposition to the disease; elevated serum cholesterol; adult-onset diabetes mellitus; elevated baseline hippocampal volume; elevated cerebrospinal fluid levels of total tau; elevated cerebrospinal fluid levels of phospho-tau; and lowered cerebrospinal fluid levels of Aβ(1-42).

16. The method of claim 11 wherein the growth hormone secretagogue is N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide, or pharmaceutically acceptable salt thereof.

17. The method of claim 11 wherein the p38 kinase inhibitor is a compound of formula XI: 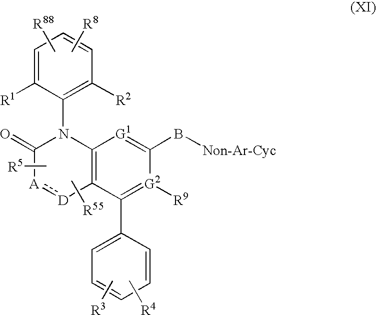
or pharmaceutically acceptable salts thereof, wherein
Non-Ar-Cyc is 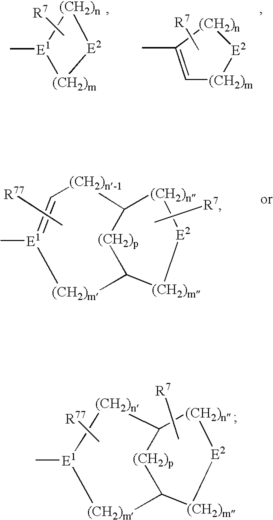
A is N, O, NH, CH₂, or CH;
B is —C₁₋₆alkyl-, —C₀₋₃alkyl-O—C₀₋₃alkyl-, —C₀₋₃alkyl-NH—C₀₋₃alkyl-, —C₀₋₃alkyl-NH—C₃₋₇cycloalkyl-, —C₀₋₃alkyl-N(C₀₋₃alkyl)-C(O)—C₀₋₃alkyl-, —C₀₋₃alkyl-NH—SO₂—C₀₋₃alkyl-, —C₀₋₃alkyl-, —C₀₋₃alkyl-S—C₀₋₃alkyl-, —C₀₋₃alkyl-SO₂—C₀₋₃alkyl-, —C₀₋₃alkyl-PH—C₀₋₃alkyl, —C₀₋₃alkyl-C(O)—C₀₋₃alkyl, or a direct bond;
D is CH, CH₂, N, or NH; optionally A and D are bridged by —C₁₋₄alkyl- to form a fused bicyclo ring with A and D at the bicyclo cusps;
E¹ is CH, N, or CR⁶; or B and E¹ form —CH═C<;
E² is CH₂, CHR, C(OH)R NH, NR, O, S, —S(O)—, or —S(O)₂—;
G¹ is N, CH, or C(C₁₋₃alkyl);
G² is N, CH, or C(C₁₋₃alkyl);
R, R⁷ and R⁷⁷ each independently is hydrogen, C₁₋₆alkyl-group, C₂₋₆alkenyl-group, C₄₋₆cycloalkyl-C₀₋₆alkyl-group, N(C₀₋₄alkyl)(C₀₋₄alkyl)-C₁₋₄alkyl-N(C₀₋₄alkyl)-group, —N(C₀₋₄alkyl)(C₀₋₄alkyl) group, C₁₋₃alkyl-CO—C₀₋₄alkyl-group, C₀₋₆alkyl-O—C(O)—C₀₋₄alkyl-group, C₀₋₆alkyl-C(O)—O—C₀₋₄alkyl-group, N(C₀₋₄alkyl)(C₀₋₄alkyl)-(C₀₋₄alkyl)C(O)(C₀₋₄alkyl)-group, phenyl-C₀₋₄alkyl-group, pyridyl-C₀₋₄alkyl-group, pyrimidinyl-C₀₋₄alkyl-group, pyrazinyl-C₀₋₄alkyl-group, thiophenyl-C₀₋₄alkyl-group, pyrazolyl-C₀₋₄alkyl-group, imidazolyl-C₀₋₄alkyl-group, triazolyl-C₀₋₄alkyl-group, azetidinyl-C₀₋₄alkyl-group, pyrrolidinyl-C₀₋₄alkyl-group, isoquinolinyl-C₀₋₄alkyl-group, indanyl-C₀₋₄alkyl-group, benzothiazolyl-C₀₋₄alkyl-group, any of the groups optionally substituted with 1-6 substituents, each substituent independently being —OH, —N(C₀₋₄alkyl)(C₀₋₄alkyl), C₁₋₄alkyl, C₁₋₆alkoxyl, C₁₋₆alkyl-CO—C₀₋₄alkyl-, pyrrolidinyl-C₀₋₄alkyl-, or halogen;
or R⁷ together with a bond from an absent ring hydrogen is ═O;
n′+n″=n;
m′+m″=m;
n is 1, 2, 3, or 4;
m is 0, 1, 2, 3, or 4;
n+m is 2, 3, 4, 5, or 6;
p is 0, 1, 2, or 3;
R¹, R², R³, R⁴, and R⁶ are each independently halogen, C₀₋₄alkyl, —C(O)—O(C₀₋₄alkyl), or —C(O)—N(C₀₋₄alkyl)(C₀₋₄alkyl);
R⁵ and R⁵⁵ independently is H, CH₃, CH₂CH₃, or absent;
R⁸⁸ and R⁸ each is independently —CN, —C₀₋₄alkyl, —C(O)—N(C₀₋₄alkyl)(C₀₋₄alkyl), —C(O)—O—C₀₋₄alkyl or 1,3-dioxolan-2-yl-C₀₋₄alkyl-;
R⁹ is —C₀₋₄alkyl, or absent; and
any alkyl is optionally substituted with 1-6 independent halogen or —OH.

18. A pharmaceutical composition comprising a growth hormone secretagogue or a pharmaceutically acceptable salt thereof, a p38 kinase inhibitor or a pharmaceutically acceptable salt thereof and a pharmaceutically acceptable carrier.
